(19) United States
(12) Patent Application Publication
(10) Pub. No.: US 2016/0242733 A1
(43) Pub. Date: Aug. 25, 2016

(54) TISSUE LESION DETECTION AND DETERMINATION USING QUANTITATIVE TRANSMISSION ULTRASOUND

(71) Applicant: QT Ultrasound LLC, Novato, CA (US)

(72) Inventors: MARK W. LENOX, College Station, TX (US); JAMES W. WISKIN, Salt Lake City, UT (US); DAVID T. BORUP, Salt Lake City, UT (US); ELAINE IUANOW, Danvers, MA (US); JOHN C. KLOCK, Nicasio, CA (US)

(21) Appl. No.: 14/627,415

(22) Filed: Feb. 20, 2015

(51) Int. Cl. A61B 8/08; A61B 8/15; A61B 8/14

(52) U.S. Cl. CPC A61B 8/085; A61B 8/14; A61B 8/15; A61B 8/5246; A61B 8/5223; A61B 8/0825

(57) ABSTRACT

The speed of sound data corresponding to transmission of ultrasound through a cancerous lesion is different than the speed of sound data corresponding to transmission of ultrasound through a benign lesion. The system can assign a coloration to a speed of sound image according to the speed of sound through the tissue as obtained from quantitative transmission ultrasound. The shape indicative of a lesion can be identified through the reflection data with the type of lesion identifiable by the coloration from the speed of sound data.

### BACKGROUND

Breast cancer and other progressive diseases that involve growth of tissue lesions may be arrested by early detection through screening and subsequent treatment. The point at which an individual's tumor development is arrested can be crucial to prognosis. Early detection by screening can reduce the incidence of advanced tumors by detecting cancers of a smaller size. Detection of small (<10 mm) lymph-node-negative, invasive cancers may save lives and provide the patient with the opportunity for less aggressive treatment. Indeed, evidence suggests that the majority of the mortality benefit from screening derives from the detection of early stage invasive cancers.

### BRIEF SUMMARY

Techniques and systems for the detection and determination of tissue lesions are described. Speed of sound data generated by a quantitative transmission ultrasound system is used to detect and identify benign and cancerous lesions in tissue. Advantageously, certain implementations of the described techniques and systems are capable of distinguishing between benign and cancerous lesions in dense breast tissue.

In some implementations, a tissue lesion is identified through a combination of reflection data and the speed of sound data obtained from a quantitative transmission ultrasound (QTUS). The system can assign a coloration to a pixel region based on speed of sound data, and the shape indicative of a lesion can be identified through the reflection data with the type of lesion identifiable by the coloration from the speed of sound data.

In particular, the speed of sound data corresponding to transmission of ultrasound through a cancerous lesion is different than the speed of sound data corresponding to transmission of ultrasound through a benign lesion. The speed of sound data may be directly used or converted into an equivalent stiffness measure. A cyst can be identified from a rounded surface shape and a slow speed; whereas a cancerous lesion can be identified from an irregular shape and a high speed. The speed of sound image data (including coloration) and the reflection image data may be displayed together (merged) or separate to facilitate the visual identification of the existence and type of lesion in even dense breast tissue.

This Summary is provided to introduce a selection of concepts in a simplified form that are further described below in the Detailed Description. This Summary is not intended to identify key features or essential features of the claimed subject matter, nor is it intended to be used to limit the scope of the claimed subject matter.

### BRIEF DESCRIPTION OF THE DRAWINGS

FIG. 1 illustrates a topography of a Quantitative Transmission Ultrasound (QTUS) system.

FIG. 2 illustrates rotation of the QTUS system of FIG. 1.

FIGS. 3A and 3B illustrate example implementations of a QTUS system for 360° data acquisition.

FIG. 4 illustrates a system environment.

FIG. 5 illustrates a process flow diagram that can be carried out by an acquisition control system.

FIG. 6 illustrates a process flow for tissue lesion determination using data from a QTUS system.

FIG. 7A shows a merged speed of sound and reflection image of a benign cyst in a patient's breast.

FIG. 7B shows a merged speed of sound and reflection image of an invasive ductile carcinoma in a patient's breast.

FIG. 8 illustrates a process flow for tissue lesion determination using data from a QTUS system.

FIG. 9 shows an example computing system through which lesion identification may be carried out.

FIG. 10A shows a QTUS transmission image of a normal breast.

FIG. 10B identifies breast anatomy from a Patrick Lynch rendering of breast anatomy

FIGS. 11A-11F show distribution graphs for a feasibility study.

### DETAILED DISCLOSURE

Quantitative Transmission Ultrasound (QTUS) techniques and systems are provided for the detection and determination of lesions in dense tissues. In certain implementations, QTUS uses ultrasound energy to image and characterize breast tissue.

For every cancer that is detected using current imaging there may be at least 5 women who unnecessarily go through the cancer diagnostic process. Advantageously, certain implementations of the described techniques are suitable for enabling a radiologist to determine if a mammographic abnormality is potentially a problem and reduce the number of unnecessary biopsies.

Unlike digital or film mammography, the described systems and techniques can detect lesions (e.g., from cancers) in heterogeneous and dense breast parenchymal tissue. In some cases, through the described techniques, it is possible to determine if a solid lesion is growing and at what rate. Breast parenchymal density is greater in women under age 50, but may be present at all ages, and is estimated to exist in 40% to 60% of all women undergoing breast cancer screening. The sensitivity of mammography is as low as 48% in women with extremely dense breasts (>75% breast density). Mammographic breast density itself is a strong independent risk factor for developing breast cancer, with risk increasing in proportion with percentage breast density as the sensitivity of mammography declines.

A QTUS system performs both reflection and transmission ultrasound methods to gather data. The reflection portion directs pulses of sound wave energy into tissues and receives the reflected energy from those pulses—hence it is referred to as “reflection ultrasound.” Detection of the sound pulse energies on the opposite side of a tissue after it has passed through the tissue is referred to as “transmission ultrasound.”

In particular, QTUS uses inverse scatter technology providing transmission information (speed of sound and attenuation) mapping of breast tissue, which also serves as a data set to reconstruct 3D breast ultrasound images using the speed data to create a reflection image volume (a reflection image corrected for refraction by the speed of sound mapping).

QTUS enables evaluation of tissue in clinical ultrasound by offering high spatial and contrast resolution, artifact-free, with absolute spatial registration (no image warping or stretching) quantitative imaging. Advantageously, the resulting images can be used for the detection of breast cancer.

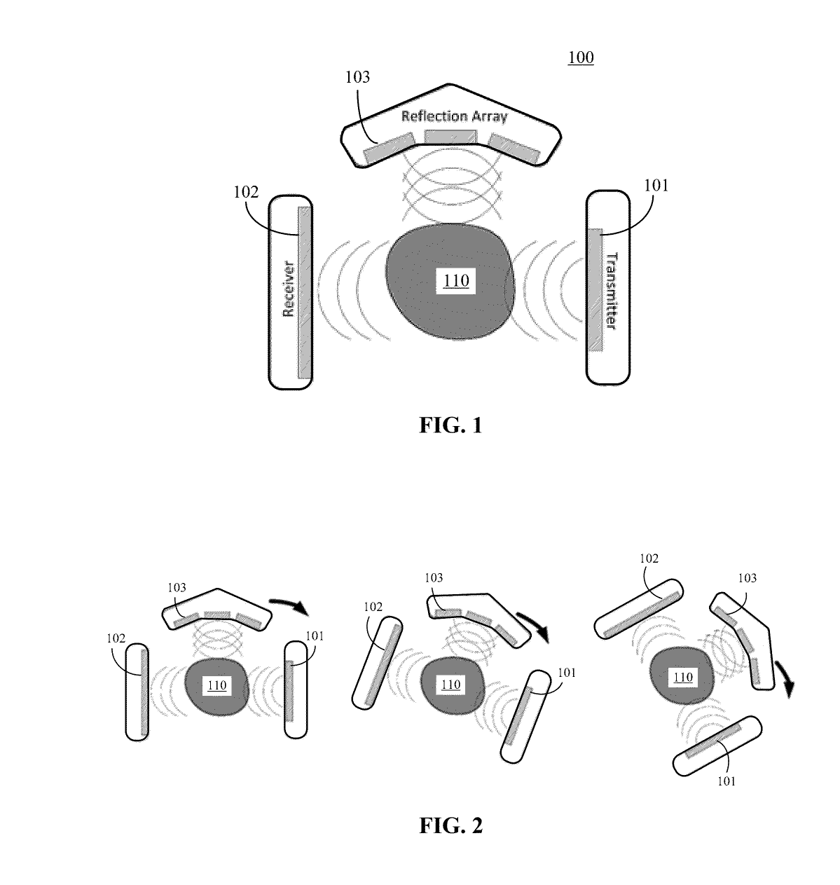

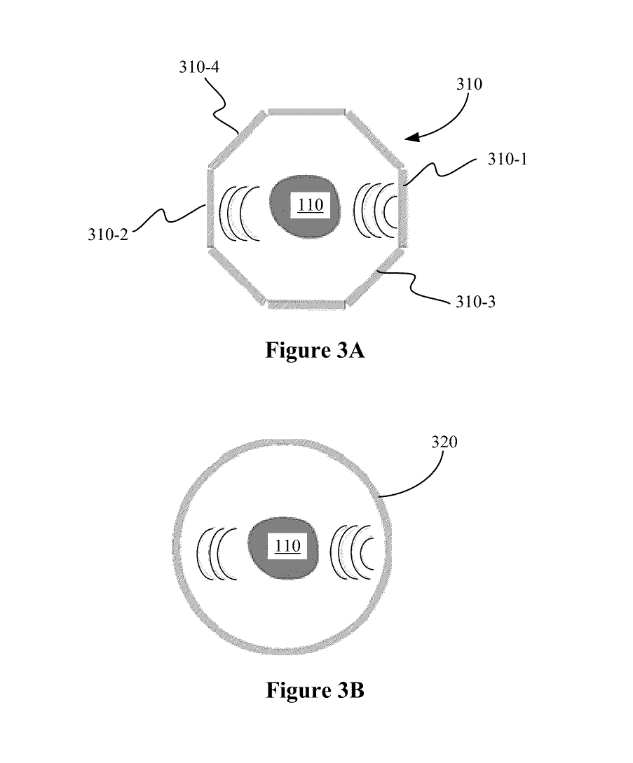

FIG. 1 illustrates a topography of a QTUS system; FIG. 2 illustrates rotation of the QTUS system of FIG. 1. Referring to FIG. 1, the imaging portion of the QTUS system 100 can include a transmitter 101, receiver 102, and transducer 103. A receptacle 110 is provided to present a water (or other liquid or gel) bath in which a patient may rest at least the region of interest (e.g., the part being imaged). The transmitter 101 and a receiver 102 are provided on opposite sides so that the receiver 102 is placed to perform transmission ultrasound. The transmitter 101 and the receiver 102 may be in the form of an array of transmitters and receivers. The transmitter array emits broad-band plane pulses (0.3-2 MHz) while the receiver array includes elements that digitize the time signal. A series of reflection transducers 103 are also included to perform reflection measurements. The reflection transducers 103 can include transducers of varying focal lengths, providing a large depth of focus when combined. The reflection imaging provides images that represent propensity for reflection information (impedance mismatch) spatially. The reflection images can be refraction-corrected and attenuation-calibrated using the speed-of-sound and attenuation information acquired from the transmission data.

As illustrated in FIG. 2, 360° of data can be obtained through rotation of the system. The system (particularly arms containing the transmitter 101 and the receiver 102) may rotate 360° to acquire measurements from effectively all the angles (e.g., data sufficient to provide a 360° view even if not taken at every angle between 0° and 360°) and collect tomographic views of ultrasound wave data. The reflection transducer data can be collected with one or more horizontal reflection transducers 103 that acquire data in steps or continuously as they rotate 360° along with the transmitter 101 and receiver 102.

In a specific implementation, the system rotates around the patient while both transmission and reflection information are captured. It is not necessary to acquire an entire 360° scan; images can be reconstructed with missing information. For example, a patient can lie prone with their breast pendent in a controlled temperature water bath (e.g., 31° C.) within the field of view of the transmitter 101 and receiver 102 as the transmitter 101, receiver 102, and transducer 103 rotates 360° around the patient. Then, in one example case 180 tomographic views of ultrasound wave data may be obtained. In another example case, 200 to up to 360 tomographic views of the ultrasound wave data may be obtained.

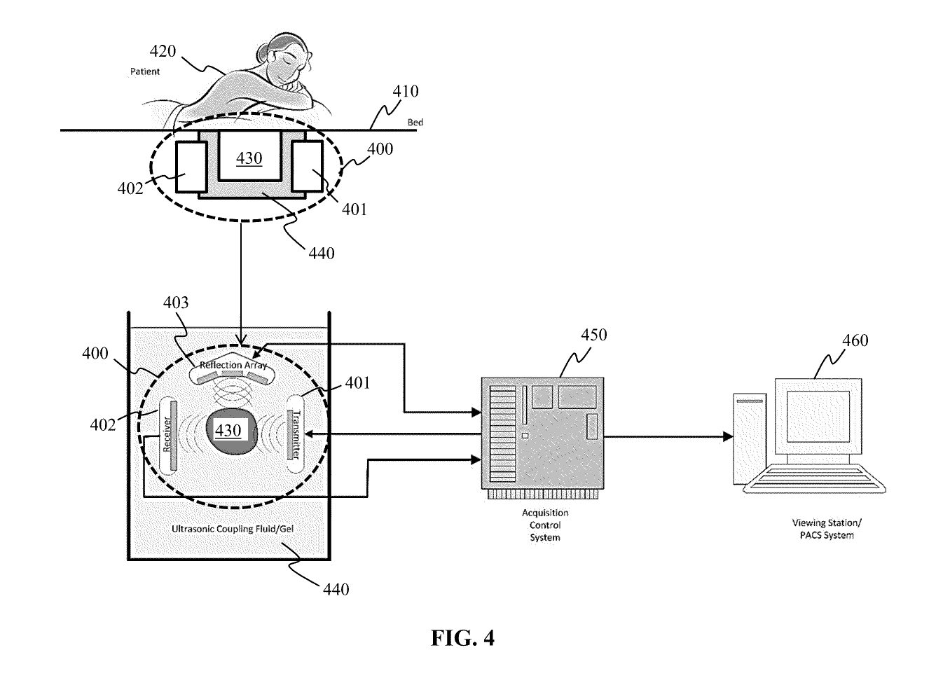

Other detector configurations may be used. For example, additional detectors in a continuous or discontinuous ring or polygon configurations (see e.g., FIGS. 3A and 3B) may be used. Of course, any configuration selected will have tradeoffs in speed and cost. In addition, in some cases, reflection arrays (the transducers for the reflection measurements) can do double-duty and perform independent transmission and receiver functions as well as reflection measurements.

FIGS. 3A and 3B illustrate example implementations of a QTUS system for 360° data acquisition. As illustrated in FIGS. 3A and 3B, 360° of data can be obtained through a fixed configuration of receivers, transmitters, and transceivers. That is, instead of rotating a same array of receivers, transmitters, and transceivers around the patient, the receivers, transmitters, and transceivers can be fixedly arranged around the patient and activated in segments to obtain 360° of data. Referring to FIG. 3A, a plurality of segments 310 are arranged around receptacle 110. Each segment can contain a transmitter array, a receiver array, and a transceiver array. In some cases, each segment can simply contain a transceiver array that is controllably operated to provide the transmitter, receiver and transceiver configurations for collecting the transmission and the reflectance data.

In an example operation, a first segment 310-1 can be operated as a transmitter and a second segment 310-2 opposite the first segment 310-1 can be operated as a receiver. Then, the first segment 310-1 can be deactivated and a third segment 310-3 can be operated as a transmitter with a fourth segment 310-4 operated as a receiver. The first segment 310-1 can also be operated as a transceiver to obtain reflectance information. The reflectance information can be obtained by operating the first segment 310-1 to both transmit and receive data (as a transceiver) before or after operation (and data collection) of the transmission signal from the first segment 310-1. The collection pattern may be any suitable pattern for collection. For example, in some cases, all of transmission data is collected and then all of the reflectance data is collected (or vice versa). In some cases, the transmission and reflectance information is alternatingly collected. For example, the same first segment 310-1 may be operated first for transmission ultrasound and second for reflection ultrasound (or vice versa). The segments 310, depending on implementation, are operated sequentially clock-wise, sequentially counter clock-wise, random, or any other suitable collection pattern so long as both transmission and reflectance information is obtained. Spacing between segments may be negligible or may have millimeter, centimeter, or inches of space, depending on implementation.

In the example shown in FIG. 3B, instead of a plurality of segments 310, a continuous or circular platform 320 may be used. Similar to the segments described with respect to FIG. 3A, the platform may contain arrays of transceivers. The transceivers may be single purpose devices where some devices perform only as receivers (as a receiver array), other devices perform only as transmitters (as a transmitter array), and other transmitter and receivers perform only for the reflectance operations (as a transceiver array). In other cases, the transceivers can perform multiple duties—where the same device is used to perform the transmission and reflectance operations. In either case, selected transmitters, receivers, and transceivers (for reflectance measurements) may be operated to obtain 360° of data around the receptacle 110.

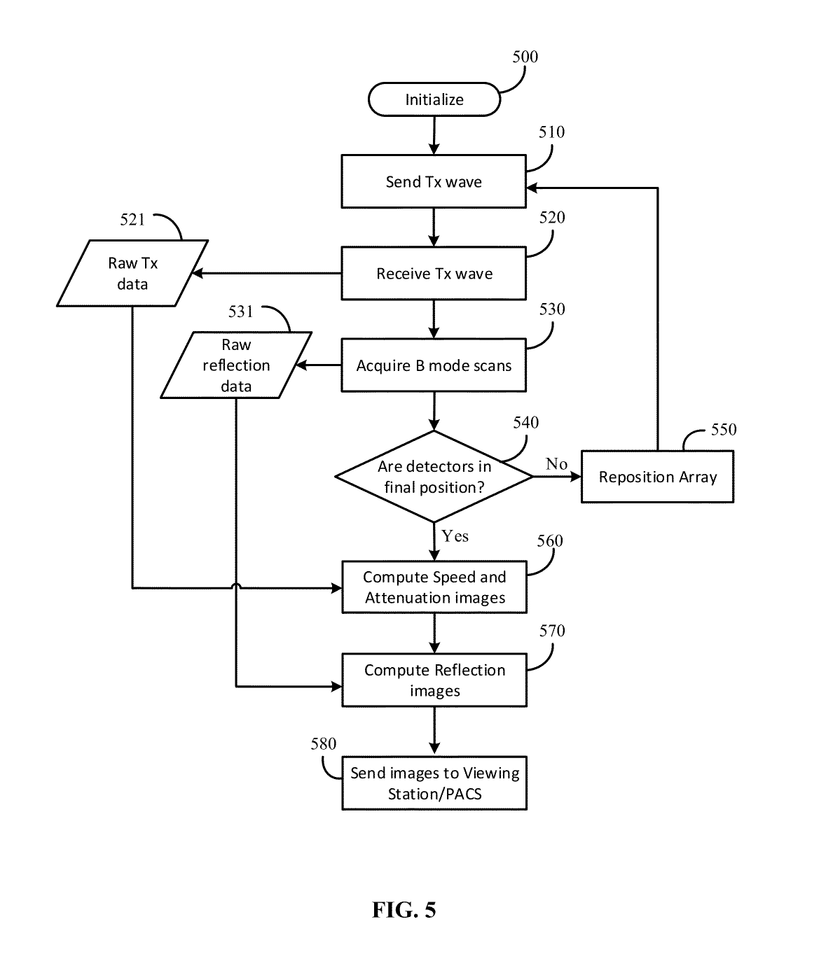

FIG. 4 illustrates a system environment. Referring to FIG. 4, active components (e.g., the imaging components, or transducers, of a QTUS system 400), such as a transmitter 401, receiver 402, and reflection array 403, can be disposed around a receptacle 430 beneath a bed 410 on which a patient 420 can lie. The patient 420 can be scanned in the prone position, resulting in a comfortable procedure.

The active components (transducers of QTUS system 400) are arranged so that data may be obtained 360° around the receptacle 430 in the bed 410 (via any suitable configuration including those shown in FIGS. 2, 3A, and 3B); and are coupled to the patient with an ultrasonic coupling medium 440 (fluid or gel), at least some of which is disposed in the receptacle 430. An acquisition control system 450 operates the various active components (e.g., the transducers) and can control their physical motion (when system 400 is arranged in a rotating configuration).

The acquisition control system 450 can automate a scan in response to a start signal from an operator. This automated acquisition process does not require operator interaction during the scanning procedure. Once the scan is complete, the acquisition control system 450 (or other computing system having access to the data) can compute the reflection, speed, and attenuation results from the collected data. The acquisition protocol enables temporal comparisons of 3D data sets; and these data sets can be compared in the same plane and orientation as those acquired with other 3D modalities, such as magnetic resonance imaging (MRI). The acquisition control system 450 can transmit the results to a viewing station 460 and/or a picture archival and communication system (PACS). Thus, images can be automatically acquired, stored for processing, and available for physician review and interpretation at the review workstation 460.

PACS refers to the software tools and environment providing storage, retrieval management, distribution, and presentation of medical images. PACS software provides an access point across multiple sites and platforms to the imaging (and imaging-related) data stored on one or more storage systems managed by the PACS software. The PACS' storage systems contain archives for storage and retrieval of images, documentation, and reports, which are generally accessed via secure networks. One or more secure networks (e.g., intranet, local area network, wide area network, etc.) are used to communicate between various devices and computing systems. The PACS generally includes and/or communicates with one or more imaging systems such as ultrasound systems, magnetic resonance imaging (MRI) systems and computed tomography (CT) scan equipment; and one or more computing devices (including workstations and portable computing devices).

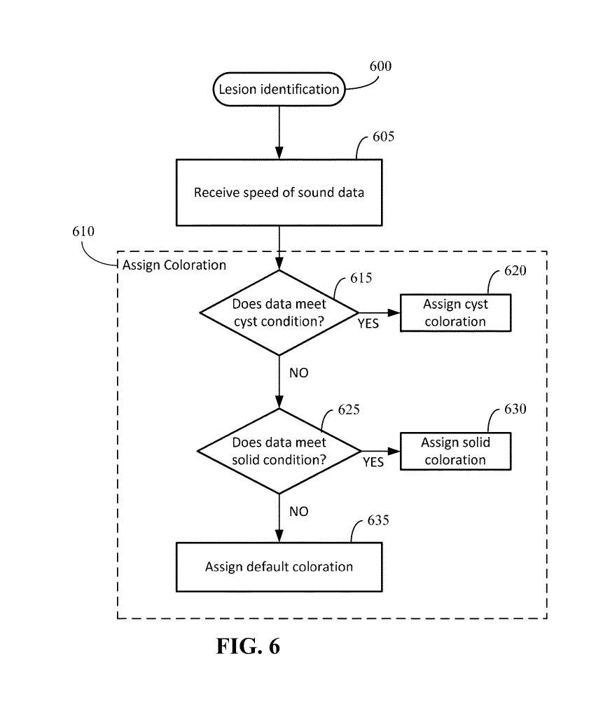

FIG. 5 illustrates a process flow diagram that can be carried out by an acquisition control system. In response to receiving an indication to initiate automated scanning (e.g., from an operator), an acquisition control system, such as system 450, can initialize (500) and send a transmission wave from a specified angle about a patient (510), for example from one or more transmitters (such as transmitter 401). As the receiver(s) 402 sense the signal transmitting through the patient (520), raw transmission data 521 is captured. Then, B mode scans, for example using transceivers 403, are acquired (530) to obtain raw flection data 531. Of course, in some cases, the B mode scans may be performed before the transmission ones.

The acquisition control system determines whether the detectors are in the final position (540). For a rotating system, such as shown in FIG. 4 (and FIGS. 1 and 2), the acquisition control system can communicate with a motor control of the platform on which the active components are provided so that a current and/or next position of the platform is known and able to be actuated. For a fixed system, the acquisition control system determines the selection of the active arrays according to an activation program. Accordingly, the “detection” of final position may be based on information provided by the motor control, position sensors, and/or a position program (e.g., using counter to determine whether appropriate number of scans have been carried out or following a predetermined pattern for activating transceivers). If the detectors are not in final position, the acquisition control system causes the array to be repositioned (550), for example, by causing the platform to rotate or by selecting an appropriate array of transceivers of a fixed platform configuration. After the array is repositioned, the transmission wave is sent (510) and received (520) so that the raw transmission data 521 is collected and the B mode scans can be acquired (530) for raw reflection data 531. This repeats until the detectors are determined to be in the final position.

Once all the data is collected (and the detectors completed the final position), speed and attenuation images are computed (560) using the raw transmission data 521, and reflection images are computed (570) using the raw reflection data 531. The reflection images are corrected for refraction with the aid of the speed of sound images. In some cases, both the original uncorrected reflection images and the refraction corrected reflection images may be available and sent to a viewing station and/or PACS (e.g., systems 460 of FIG. 4) (580).

The refraction correction reflection images and the speed of sound images can then be used to determine whether a benign or cancerous lesion is present regardless of tissue density. The speed of sound information provides information about the type of breast tissue traversed. The information about the type of breast tissue, in combination with additional parameters, such as surface-to-volume ratio and doubling time, provides more accurate, specific information regarding a breast lesion(s), thus improving accuracy and potentially decreasing unnecessary biopsies of benign lesions.

Reflection images can show the surface shape of a lesion; whereas transmission images may present information about the structure behind the surface. In general, a cyst has a more even and spherical shape as it is usually a sac-like structure filled with liquid, gas, or semi-solid material. In contrast, a cancerous lesion or tumor has a less uniform shape and is usually harder and denser than a cyst since a tumor is formed of a mass of tissue. Although not all tumors are cancerous and not all cysts are benign, being able to detect and track the growth of tumors and cysts can improve diagnosis and potentially reduce unnecessary biopsies of benign lesions.

A technique is described herein that uses speed of sound maps computed from the transmission information of the QTUS system to aid in the identification of normal breast tissue, benign lesions, and cancerous lesions.

Here, the speed of sound information provides a quantitative number corresponding to the density of a tissue region. Even in dense breast tissue, the density characteristics of a cyst and a tumor are sufficiently different so that the type of lesion detected in a breast can be identified. The speed of sound maps are used on their own for diagnosis (characterization of benign versus cancerous lesions) and for correcting reflection images for refraction effects. Both the speed of sound maps and reflection images can be provided to a practitioner.

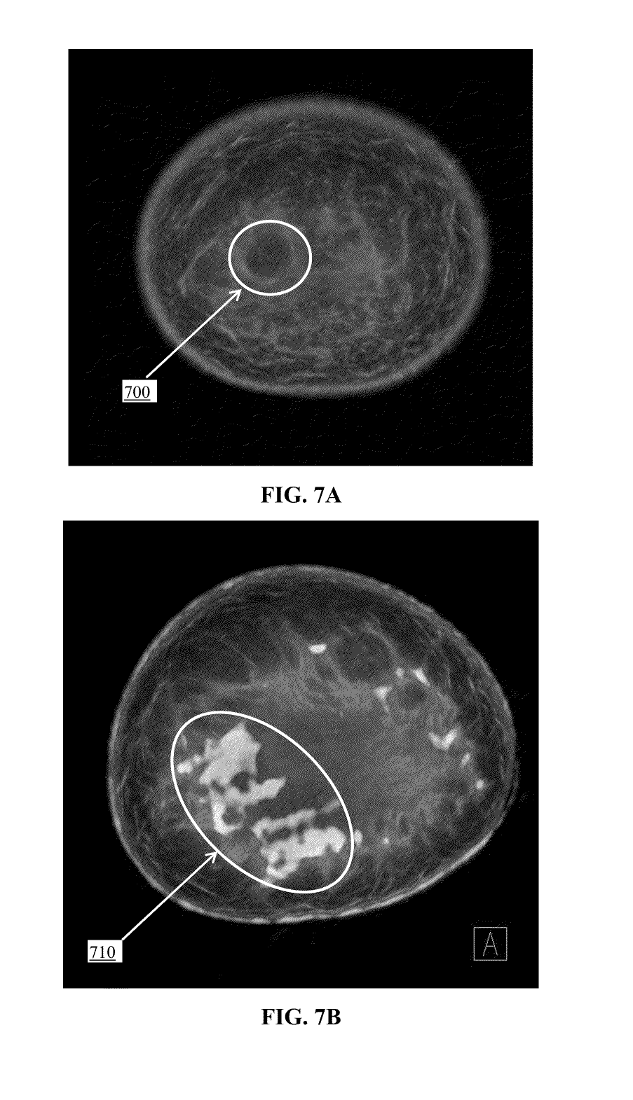

FIG. 6 illustrates a process flow for tissue lesion determination using data from a QTUS system. Referring to FIG. 6, lesion identification (600) can be carried out upon receipt of speed of sound data from a QTUS system (605). In some cases, this process may be carried out during the computing of speed and attenuation images (560) of FIG. 5. A pixel is assigned a coloration based on the speed of sound data for that pixel (610). The coloration refers to data associated with color properties so that when the information from the speed of sound data is transformed into an image, the color properties are reflected in the image. Color properties can be hue, intensity, and/or any other recognizable property of color. This color property is used to generate a speed of sound image and can be in any suitable format for rendering or otherwise displaying the image.

In one implementation, coloration is assigned purely according to the speed information so that a lower speed is assigned a characteristic from one end of a spectrum while a higher speed is assigned a characteristic from the other end of the spectrum for a particular color property.

In another implementation, as illustrated in FIG. 6, an additional level of processing may be carried out. For example, the assigning of coloration (610) can include determining whether the data meets a cyst condition (615). If the data does meet the conditions for likely being a cyst, cyst coloration is assigned (620). The assigning of coloration (610) can further included determining whether the data meets a solid condition (625). If the data does meet the conditions for likely being a solid, solid coloration is assigned (630). If the data does not meet the conditions for being a cyst or meet the conditions for being a solid, a default coloration may be applied (635).

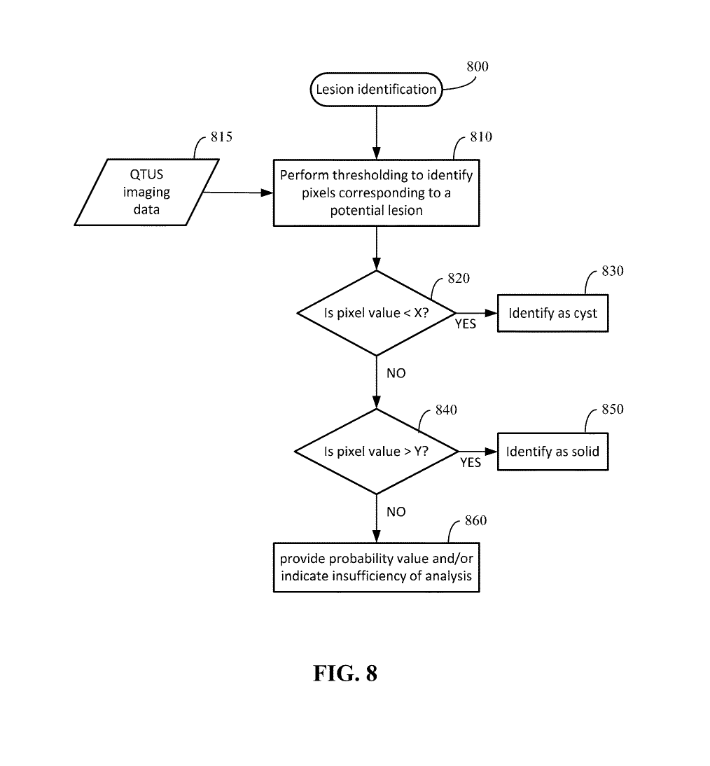

FIG. 7A shows a merged speed of sound and reflection image of a benign cyst in a patient's breast; and FIG. 7B shows a merged speed of sound and reflection image of an invasive ductile carcinoma in a patient's breast. As can be seen in FIG. 7A, a round lesion is identifiable from the reflection image portion of the merged image and a coloration from the speed of sound image portion shows a coloration indicative of the slow speed of a cyst 700. In a specific implementation from which the image was obtained, the coloration for the speed indicative of a cyst is a red-like color. In contrast, as can be seen in FIG. 7B, the reflection image captures the irregular shape of a cancerous lesion and the speed of sound coloration indicates the high speed of a solid material to indicate that the lesion is a carcinoma 710. The coloration in this specific implementation is bright yellow and white.

There may be a spectrum of colors assigned to the speed values and/or a same color but different intensity (corresponding to the speed values) may be used.

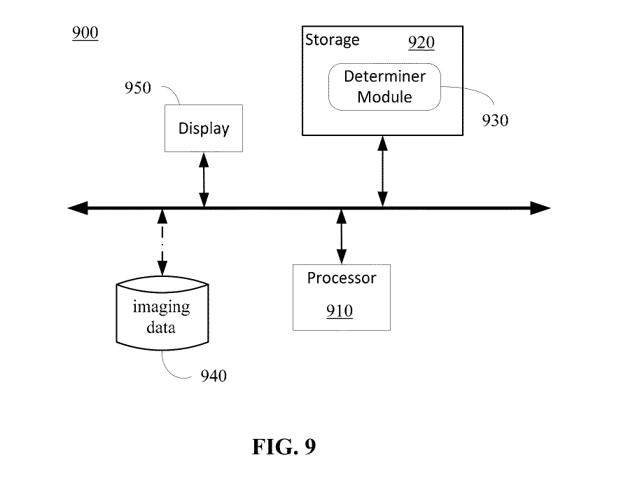

FIG. 8 illustrates a process flow for tissue lesion determination using data from a QTUS system. Referring to FIG. 8, a method of lesion identification 800 can be carried out at a computing device. Process 800 can include a thresholding process that identifies pixels corresponding to a potential lesion (810). The thresholding can be any suitable thresholding algorithm including, but not limited to, histogram, clustering, entropy, object attribute, spatial or local. The thresholding can help identify tissue regions of non-normal tissue. The thresholding can involve QTUS imaging data 815 and, in some cases, baseline data and/or known anatomy and pathology.

In some cases, a specific step to determine whether a lesion exists (e.g., operation 810) is omitted and the lesion identification process begins with a comparison process where speed of sound data (speed of sound and/or attenuation values) of the QTUS imaging data 815 obtained from an imaging cycle of a patient is compared to a reference value or range of values.

Whether or not the operation 810 is omitted, to identify whether a pixel corresponds to a region having a cyst or a solid lesion, the speed of sound value is compared with a first value X (820). If the speed of sound value is less than X, then the pixel is identified as part of a cyst (830). If the speed of sound value is not less than X, then the speed of sound value is compared with a second value Y (840). If the speed of sound value is greater than Y, the pixel is identified as part of a solid (850). However, if the speed of sound value is not greater than Y, an output or other indication may be made representative of not being able to identify with a predetermined probability that a pixel belongs to a region with a cyst or a solid (860). The identification of a pixel as belonging to a cyst region or a solid region may be presented as a number (e.g., a value indicative of a likelihood of being a cyst or solid) and/or assigned a particular coloration value and presented in an image according to the coloration (e.g., as described with respect to FIG. 6).

It should be understood that operation 820 could be performed after operation 840 in some implementations. In addition, although only two comparison steps are shown in this example, it should be understood that more or less steps may be carried out to provide different levels of granularity for the identification of a cyst or solid. For example, the speed of sound value may be compared with a table of values to find a closest matching value and corresponding probability of being a cyst or solid, and that probability provided as an output of the lesion identification process.

For the values of X and Y (or any other comparison steps), these values can be generated for the particular tissue being imaged. An initial study of breast tissue using speed of sound measurements from QTUS provided a statistical probability of >97% solid if the lesion has a speed of greater than 1571 m/s and a statistical probability of >97% cystic if the lesion has a speed of less than 1569 m/s. Using this data, for breast lesion identification, X may be given as 1569 m/s and Y may be given as 1571 m/s. As another cyst condition criteria, based on the same study, a statistical probability of >99% involves a speed equal to or less than 1530 m/s; and a another solid condition criteria, a statistical probability of >99% involves a speed equal to or greater than 1581 m/s. Similar conditions may be used for operations 615 and 625 described with respect to FIG. 6. A more detailed explanation for these values is provided in the Speed of Sound Measurements Example described below.

In addition (or as an alternative) to using the speed data directly, the speed of sound can be used to generate a value corresponding to the stiffness of the material through which the sound is traveling. In particular, since speed=\sqrt{stiffness/density}, the speed data can be used to generate stiffness values. The stiffness values (or other equivalent stiffness measure) may be used to differentiate between cyst and solids.

Process flow 500 described with respect to FIG. 5, process flow 600 described with respect to FIG. 6, and process flow 800 described with respect to FIG. 8 may be implemented in the form of computer-executable instructions, such as program modules, that are executed by one or more computers or other devices.

In some embodiments, the machine/computer system can operate as a standalone device. In some embodiments, the machine/computer system may be connected (e.g., using a network) to other machines. In certain of such embodiments, the machine/computer system may operate in the capacity of a server or a client user machine in server-client user network environment, or as a peer machine in a peer-to-peer (or distributed) network environment.

The machine/computer system can be implemented as a desktop computer, a laptop computer, a tablet, a phone, a server, or any other machine capable of executing a set of instructions (sequential or otherwise) that specify actions to be taken by that machine, as well as multiple machines that individually or jointly execute a set (or multiple sets) of instructions to perform any one or more of the methods described herein.

The computer system can have hardware including one or more central processing units (CPUs) and/or digital signal processors (DSPs), memory, mass storage (e.g., hard drive, solid state drive), I/O devices (e.g., network interface, user input devices), and a display (e.g., touch screen, flat panel, liquid crystal display, solid state display). Elements of the computer system hardware can communicate with each other via a bus.

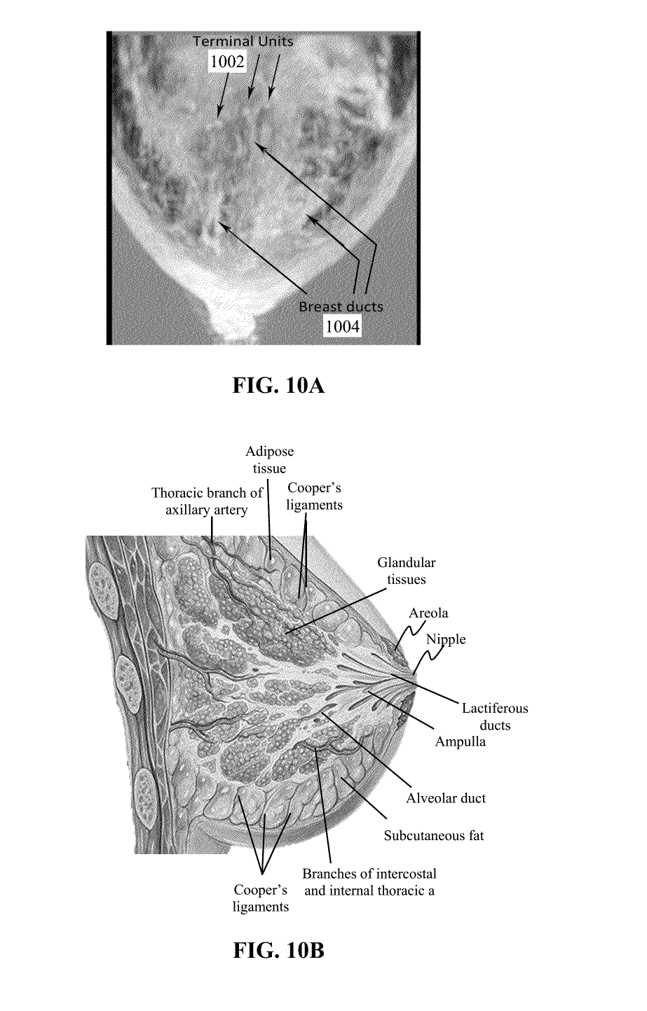

For example, FIG. 9 shows an example computing system through which lesion identification may be carried out. In some implementations, the computing system may be embodied, at least in part, as a viewing station and/or PACS. In some implementations, the computing systems may embody, at least in part, the acquisition control system. Referring to FIG. 9, the system 900 can include a processor 910 and a storage system 920 in which a lesion determiner module 930 may be stored. The lesion determiner module may carry out process 600 and/or process 800 such as described with respect to FIGS. 6 and 8. Examples of processor 910 include general purpose central processing units, application specific processors, and logic devices, as well as any other type of processing device, combinations, or variations thereof. The processor 910 processes data according to instructions of the lesion determiner module 930.

Storage system 920 includes any computer readable storage media readable by the processing system 920 and capable of storing software, including lesion determiner module 930. Storage system 920 may include volatile and nonvolatile, removable and non-removable media implemented in any method or technology for storage of information, such as computer readable instructions, data structures, program modules, or other data. Examples of storage media include random access memory (RAM), read only memory (ROM), magnetic disks, optical disks, CDs, DVDs, flash memory, solid state memory, phase change memory, or any other suitable storage media. Certain implementations may involve either or both virtual memory and non-virtual memory. In no case do storage media consist of a propagated signal or carrier wave. In addition to storage media, in some implementations, storage system 920 may also include communication media over which software may be communicated internally or externally.

Storage system 920 may be implemented as a single storage device but may also be implemented across multiple storage devices or sub-systems co-located or distributed relative to each other. Storage system 920 may include additional elements, such as a controller, capable of communicating with processor 910.

A database 940 storing speed of sound, reflection, and other imaging data from a QTUS system can be coupled to the system via wired or wireless connections.

Visual output can be provided via a display 950. Input/Output (I/O) devices (not shown) such as a keyboard, mouse, network card or other I/O device may also be included. It should be understood the any computing device implementing the described system may have additional features or functionality and is not limited to the configurations described herein.

The determiner module 930, for example, in the case of one implementation of process 800, can take advantage of the correlation of the image data to anatomy and pathology for identifying benign and cancerous lesions in breast tissue. For example as part of, or associated with, a thresholding step.

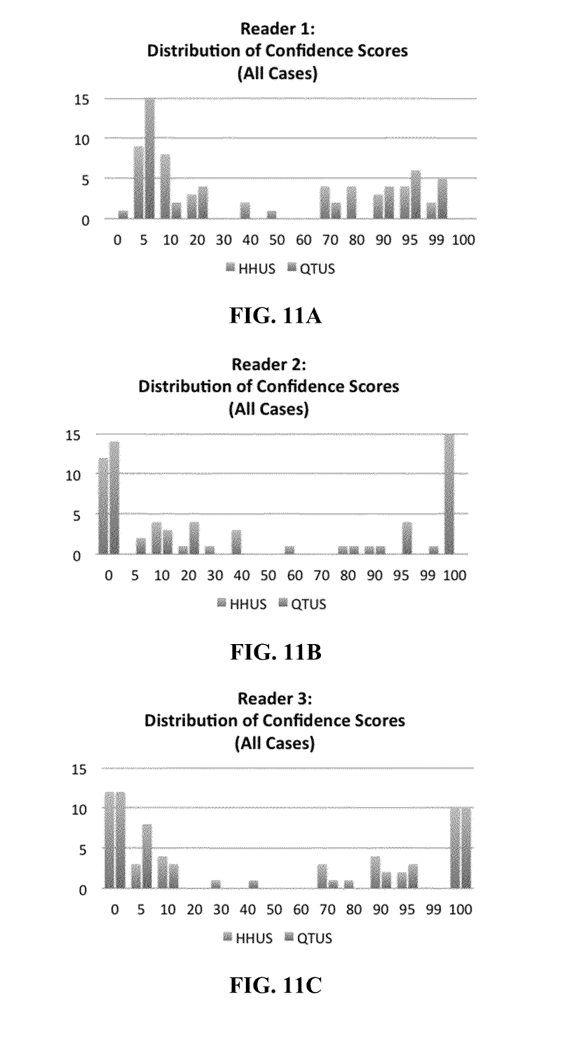

FIG. 10A shows a QTUS transmission image of a normal breast; and FIG. 10B identifies breast anatomy from a Patrick Lynch rendering of breast anatomy. Comparing the two images, it can be seen that the QTUS transmission image of a normal breast demonstrates the ductal 1002 and terminal duct 1004 lobular units in the axial plane; and shows good anatomic correlation with the artist's rendering of breast anatomy in FIG. 10B.

A greater understanding of the present invention and of its many advantages may be had from the following example, given by way of illustration. The following example is illustrative of some of the systems, methods, applications, embodiments and variants of the present invention. They are, of course, not to be considered in any way limitative of the invention. Numerous changes and modifications can be made with respect to the invention.

### Example

### Speed of Sound Measurements

Speed of sound measurements from QTUS and biopsy results were collected as part of a prospective study of consecutive patients from four sites (see Table 1). Data from 136 patients with 88 biopsy-proven cysts and 122 biopsy-proven solids (including 29 cancers) were available for this analysis.

TABLE 1

| | | #Cysts | | |
|---|---|---|---|---|
| | #Patients | in | | |
| Site | Biopsied | Analysis | #Solids in Analysis | Prevalence of Solids |
| A | 24 | 11 | 16 (6 cancers) | 0.593 |
| B | 75 | 56 | 65 (12 cancers) | 0.537 |
| C | 9 | 7 | 10 (4 cancers) | 0.588 |
| D | 28 | 14 | 31 (7 cancers) | 0.705 |
| Total | 136 | 88 | 122 (29 cancers) | 0.581 |

To measure the ability of the speed of sound measurements to discriminate solids from cysts, nonparametric methods for clustered data were used to estimate the area under the receiver operating characteristic (ROC) curve and construct a 95% Confidence Interval (CI) for the area. The ROC curve was created by plotting the true positive rate against the false positive rate. The estimated area under the ROC curve was 0.982, with 95% CI of [0.966, 0.998].

To estimate the predictive probabilities for various ranges of the speed of sound measurements, a logit (log-odds) model was fit to the data, where the dependent variable was the presence/absence of a solid and the independent variable was the speed of sound measurements. For various ranges of the speed of sound measurements, the estimated probability of a solid from the fitted model was reported, as well as the simple sample proportion. Based on these findings, a draft reader instruction sheet was constructed.

Table 2 summarizes the estimated probability of a solid, based on the fitted logit model as well as the simple sample proportion, for various ranges of the speed of sound measurements. Note that these predictive values depend on the prevalence of solids in the sample. If the prevalence rate differs from 0.581, then these estimates should be adjusted.

TABLE 2
Predictive Values

| Speed | Probability of a Cyst | | Probability of a Solid | |
|---|---|---|---|---|
| of Sound | Fitted Model | Empirical | Fitted Model | Empirical |
| Range | Estimate* | Estimate | Estimate* | Estimate |
| <1500 | >0.999 | 1.0 (1/1) | <0.001 | 0.0 (0/1) |
| 1500-1530 | >0.999 | 1.0 (7/7) | <0.001 | 0.0 (0/7) |
| 1531-1540 | 0.998 | 0.955 (21/22) | 0.002 | 0.045 (1/22) |
| 1541-1550 | 0.980 | 0.966 (28/29) | 0.020 | 0.034 (1/29) |
| 1551-1560 | 0.849 | 0.944 (17/18) | 0.151 | 0.056 (1/18) |
| 1561-1565 | 0.688 | 0.778 (7/9) | 0.312 | 0.222 (2/9) |
| 1566-1570 | 0.292 | 0.316 (6/19) | 0.708 | 0.684 (13/19) |
| 1571-1580 | 0.036 | 0.033 (1/30) | 0.967 | 0.967 (29/30) |
| 1581-1590 | 0.006 | 0.0 (0/23) | 0.994 | 1.0 (23/23) |
| 1591-1600 | <0.001 | 0.0 (0/28) | >0.999 | 1.0 (28/28) |
| >1600 | <0.001 | 0.0 (0/24) | >0.999 | 1.0 (24/24) |

*median of the fitted estimates in specified range

### Example

### Feasibility Study

Feasibility studies with the QTUS technology were performed. This technology was evaluated in the laboratory with phantom and cadaver breasts as well as IRB approved Phase I and II clinical trials on real patients. Extensive modeling was performed and compared with handheld ultrasound (HHUS) to determine the sensitivity and specificity of detecting a lesion and accurately determining whether the lesion is cyst vs. solid using the two ultrasound modalities.

Table 3 below indicates the results with 23 known targets placed in two cadaver breasts.

### Table 3

TABLE 3

| 23 Targets in 2 breasts | HHUS | QTUS |
|---|---|---|
| Measurement Accuracy | 72% | 86% |
| Sensitivity | 85% | 94% |
| Specificity | 87% | 94% |
| Positive Predictive Value | 97% | 98% |
| Negative Predictive Value | 54% | 76% |

### Example

### Three-Reader Feasibility Study

A three-reader feasibility study was performed based on the patient data acquired during phase I and II trials.

The primary objective of the study was to estimate the area under the ROC curves of X-ray mammography (XRM)+HHUS and XRM+QTUS for each of the three readers using the 0-100 confidence scale and the 1-5 ordinal scale, and to estimate the sensitivity and specificity using the binary scale. The study also estimated the area under the ROC curve of QTUS speed of sound measurements, and assessed the suitability of the 0-100 confidence score scale vs. ordinal scale vs. binary scale.

Confidence scores from 40 subjects were provided. There are 21 subjects with cysts and 19 with solid lesions. 17 lesions were in the left breast and 23 in the right.

Nonparametric estimates of the ROC areas were calculated and compared using the method of DeLong et al. “Comparing the areas under two or more correlated receiver operating characteristic curves: a nonparametric approach,” Biometrics 1988; 44: 837-845. Sensitivity was calculated as the proportion of true solids that were called a solid by the reader; specificity was calculated as the proportion of true cysts that were called a cyst by the reader.

Table 4 summarizes the estimated area under the ROC curves of XRM+HHUS and XRM+QTUS for the three readers using the 0-100 confidence scale.

TABLE 4
Nonparametric Estimates of the Area under the ROC Curve
for All Cases (n = 40) Using the 0-100 confidence score

| | XRM + HHUS | XRM + QTUS | p-value* |
|---|---|---|---|
| Reader 1 | 0.957 (0.028) | 0.970 (0.023) | 0.695 |
| Reader 2 | 0.964 (0.030) | 0.977 (0.018) | 0.688 |
| Reader 3 | 0.972 (0.020) | 0.972 (0.022) | 1.0 |
| Overall | 0.964 | 0.973 | |

*p-value testing for a difference in ROC areas between HHUS and QTUS.

Table 5 summarizes the estimated area under the ROC curves of XRM+HHUS and XRM+QTUS for the three readers using the 1-5 ordinal scale.

TABLE 5
Nonparametric Estimates of the Area under the ROC Curve
for All Cases (n = 40) Using the 1-5 ordinal score

| | XRM + HHUS | XRM + QTUS | p-value |
|---|---|---|---|
| Reader 1 | 0.930 (0.035) | 0.956 (0.036) | 0.468 |
| Reader 2 | 0.949 (0.033) | 0.987 (0.010) | 0.205 |
| Reader 3 | 0.937 (0.038) | 0.932 (0.044) | 0.925 |
| Overall | 0.939 | 0.959 | |

Readers 1 and 2 improved with QTUS (according to both scales) while reader 3 stayed the same (with the 0-100 confidence scale) or was a little worse (with the ordinal scale) with QTUS.

Note that all of the ROC areas are quite high (closer to 1.0 than expected). There was a difference in readers' mean ROC area of 0.01 with the 0-100 confidence scale and a difference of 0.02 with the 1-5 scale. The larger difference observed with the 1-5 scale could be an artifact because there are very few points on the ROC curves with this scale. The standard errors (in parentheses) are smaller using the 0-100 scale, indicating greater precision and thus more statistical power.

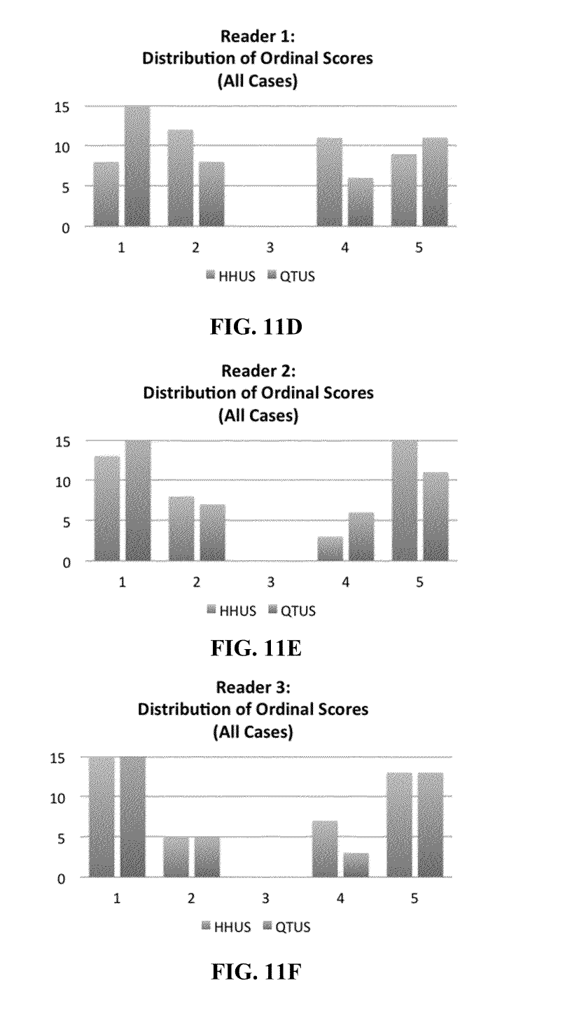

FIGS. 11A-11F illustrate the spread of the confidence and ordinal scores for each of the three readers. FIGS. 11A-11C show the distribution of confidence scores from each of the three readers of the data. FIGS. 11D-11F show the distribution of ordinal scores from each of the three readers of the data. As shown in FIGS. 11D-11F, none of the three readers used the ordinal score of 3. Thus, with the ordinal scale there are only 3 ROC operating points (i.e. three unique points on each curve). In contrast, as shown in FIGS. 11A-11C, the readers did spread out their 0-100 confidence scores allowing 7-9 ROC operating points per curve.

Table 6 summarizes the sensitivity and specificity of the three readers based on the binary scale. Two readers' sensitivities stayed the same with QTUS and one reader's sensitivity decreased with QTUS. All three readers' specificities improved with QTUS. Note that the standard errors for these binary estimates are much larger than for the area under the ROC curve.

TABLE 6
Estimates of the Sensitivity and Specificity for All Cases (n = 40)
Using the binary score

| | XRM + HHUS | XRM + QTUS | p-value |
|---|---|---|---|
| SENSITIVITY | | | |
| Reader 1 | 0.895 (0.070) | 0.895 (0.070) | 1.0 |
| Reader 2 | 0.895 (0.070) | 0.895 (0.070) | 1.0 |
| Reader 3 | 0.895 (0.070) | 0.842 (0.084) | 0.304 |
| Overall | 0.895 | 0.877 | |
| SPECIFICITY | | | |
| Reader 1 | 0.857 (0.076) | 1.0 (—) | 0.250 |
| Reader 2 | 0.905 (0.064) | 1.0 (—) | 0.500 |
| Reader 3 | 0.857 (0.076) | 1.0 (—) | 0.250 |
| Overall | 0.873 | 1.0 | |

For Table 6, “sensitivity” is defined as the proportion of lesions called “solid” by the reader among all true solids; “specificity” is defined as the proportion of lesions called “cyst” by the reader among all true cysts.

The estimated ROC area of the QTUS speed of sound measurements was 0.980 (SE=0.017) [95% CI of 0.947, 1.0]. This estimate is similar to a previous estimate from an earlier analysis, where the estimated ROC area was 0.982.

It should be understood that the examples and embodiments described herein are for illustrative purposes only and that various modifications or changes in light thereof will be suggested to persons skilled in the art and are to be included within the spirit and purview of this application.

What is claimed is:

1. A system for tissue lesion detection and determination, comprising:
one or more computer readable storage media having instructions stored thereon that, when executed by a processor, direct the processor to:
determine, for a pixel, whether speed of sound data from a quantitative transmission ultrasound (QTUS) has a speed that satisfies a cyst condition criteria or a solid condition criteria;
in response to a determination that the speed satisfies the cyst condition criteria, assign a cyst coloration for the pixel;
in response to a determination that the speed satisfies the solid condition criteria, assign a solid coloration for the pixel; and
in response to a determination that the speed does not satisfy either the cyst condition criteria or the solid condition criteria, assign a default coloration for the pixel.

2. The system of claim 1, wherein the speed satisfying the cyst condition criteria comprises the speed less than 1569 m/s; and the speed satisfying the solid condition criteria comprises the speed greater than 1571 m/s.

3. The system of claim 1, wherein the speed satisfying the cyst condition criteria comprises the speed less than or equal to 1530 m/s; and the speed satisfying the solid condition criteria comprises the speed greater than or equal to 1581 m/s.

4. The system of claim 1, further comprising instructions that direct the processor to:
identify, from imaging data including reflection data generated by the QTUS, pixels corresponding to a potential lesion.

5. The system of claim 4, wherein the instructions that direct the processor to identify pixels corresponding to the potential lesion comprise instructions to perform a thresholding process.

6. A method for tissue lesion detection and determination, comprising:
identifying a region in a reflection image generated from a quantitative transmission ultrasound (QTUS) having a shape indicative of a potential lesion; and
determining whether the potential lesion is likely a cyst or a cancerous lesion using speed of sound image data generated from the QTUS.

7. The method of claim 6, wherein the determining whether the potential lesion is likely the cyst or the cancerous lesion comprises:
determining the likely cyst by identifying the shape of a round lesion from the reflection image and a coloration from the speed of sound image indicating a speed satisfying a cyst condition.

8. The method of claim 7, wherein the speed satisfying the cyst condition comprises the speed less than 1569 m/s.

9. The method of claim 7, wherein the speed satisfying the cyst condition comprises the speed less than or equal to 1530 m/s.

10. The method of claim 6, wherein the determining whether the potential lesion is likely the cyst or the cancerous lesion comprises:
determining the likely cancerous lesion by identifying the shape of an irregular lesion from the reflection image and a coloration from the speed of sound image indicating a speed satisfying a solid condition.

11. The method of claim 10, wherein the speed satisfying the solid condition comprises the speed greater than 1571 m/s.

12. The method of claim 10, wherein the speed satisfying the solid condition comprises the speed greater than or equal to 1581 m/s.

13. The method of claim 6, wherein the speed of sound image data is first converted to a stiffness value before determining whether the potential lesion is likely the cyst or the cancerous lesion.

14. One or more computer readable storage media having instructions stored thereon that, when executed by a processor, direct the processor to:
receive speed of sound image data and reflection image data from a quantitative transmission ultrasound (QTUS);
perform a thresholding of at least one of the speed of sound image data and the reflection image data to identify pixels corresponding to a potential lesion;
for at least one pixel of the pixels corresponding to the potential lesion, determine whether the pixel's speed of sound data has a speed that satisfies a cyst condition criteria or a solid condition criteria;
in response to a determination that the speed satisfies the cyst condition criteria, identify the pixel as having a likelihood of being a cyst; and
in response to a determination that the speed satisfies the solid condition criteria, identify the pixel as having a likelihood of being a solid.

15. The media of claim 14, wherein the speed satisfying the cyst condition criteria comprises the speed less than 1569 m/s; and the speed satisfying the solid condition criteria comprises the speed greater than 1571 m/s.

16. The media of claim 14, wherein the speed satisfying the cyst condition criteria comprises the speed less than or equal to 1530 m/s; and the speed satisfying the solid condition criteria comprises the speed greater than or equal to 1581 m/s.
